(19) United States
(12) Patent Application Publication
(10) Pub. No.: US 2002/0071876 A1
(43) Pub. Date: Jun. 13, 2002

(54) Method of removing accumulated substances from the body

(76) Inventor: Russell D. Briglia, San Diego, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/020,595

(22) Filed: Dec. 7, 2001

Related U.S. Application Data
(60) Provisional application No. 60/254,999, filed on Dec. 11, 2000.

(51) Int. Cl. A61K 35/78

(52) U.S. Cl. 424/725.1; 424/726; 424/745; 424/757; 424/773

(57) ABSTRACT

A method of removing pharmacologically active substances and/or their metabolites from the body by administering material having the physiological activity of Psyllium husk, Golden Seal root, beet root, activated charcoal, and one of the following: Chaparral leaves or flowers, Thisilyn, or Chaparral root at timed intervals over a two to ten day period. This method is preferably accompanied by the administration of relatively pure water.

### CROSS-REFERENCE TO RELATED APPLICATION

This application claims priority to and incorporates by reference in its entirety, U.S. Provisional Application No. 60/254,999, filed Dec. 11, 2000.

### BACKGROUND OF THE INVENTION

### FIELD OF THE INVENTION

The present invention relates generally to a method of removing pharmaceutically active compounds and metabolites from the human body, reducing the risk of poisoning or decreasing the persistence of those compounds in the body, thereby reducing side effects. More specifically, the invention relates to a combination of herbs and natural products introduced to the body, preferably with ample amounts of water, at intervals effective to rid the body of unwanted chemical substances.

### SUMMARY OF THE INVENTION

The present invention relates to a method of removing pharmaceutically active compounds from the body. Certain aspects of society are more comfortable with herbal products and remedies, and indeed, science is increasingly demonstrating that such products contain natural active ingredients that provide beneficial effects when properly used.

One aspect of the present invention is a method of reducing the amount of an exogenous pharmacologically-active compound or its metabolite from the body, comprising: administering an effective dose of: at least two liver/blood/kidney cleansers each having the pharmacological activity of at least one of Chaparral, Thisilyn, beet root, Yellow Dock, Clover, Rosemary, Golden Seal, and/or Yucca, a lower gastrointestinal tract cleanser having the pharmacological activity of beet root; and an adsorbent material. In one preferred embodiment, the liver/blood/kidney cleansers are selected from the group consisting of Golden Seal root, Golden Seal Herb, beet root, Chaparral leaves or flowers, Thisilyn, Chaparral root, Yellow Dock (Rumex crispus) (root), Red Clover (Trifolium pratense) (blossom), Rosemary (Rosmarinus officinalis) (aerial), and Yucca (Yucca schidigera) (stalk). Preferably, the lower gastrointestinal tract cleanser comprises beet root. In a preferred embodiment, the adsorbent material comprises charcoal, preferably activated charcoal.

The present invention also includes a method of reducing the amount of an exogenous pharmacologically-active compound or its metabolite from the body, comprising administering to a subject an effective amount of: Golden Seal root; one of the following: Chaparral leaves or flowers, Thisilyn, or Chaparral root; beet root; and charcoal. Preferably, the charcoal comprises activated charcoal. A preferred method for administration is by oral ingestion. The method preferably also includes administration of a bulk former, such as soluble or insoluble dietary fiber, preferably Psyllium fiber. It is preferred to perform the administration step at a multi-hourly interval for a period of about two to ten days. Preferably, the interval ranges from about 2 to about 12 hours, more preferably about 6 hours. In one preferred embodiment, the method further includes giving the subject a volume of water in conjunction with the administration step. Preferred types of water include distilled water, bottled water, and demineralized water. The volume of water preferably comprises at least about 8, 10, 12, 16, or 20 ounces per dose, and more preferably about 30, 32, 36, or 40 ounces per dose. Preferably, the dose of Golden Seal root is about 200 milligrams to about 700 milligrams, more preferably about 535 milligrams. The dose of Chaparral or Thisilyn is preferably about 120 milligrams to about 800 milligrams, more preferably about 450 milligrams. The dose of beet root is preferably about 250 milligrams to about 900 milligrams, more preferably about 600 milligrams. Finally, the dose of charcoal is preferably about 50 milligrams to about 600 milligrams, more preferably about 280 milligrams.

Another aspect of the present invention is a method of removing physiologically active endogenous substances or their metabolites from the body comprising the steps of: first, administering Psyllium fiber, Golden Seal root, beet root, activated charcoal, and one of the following: Chaparral leaves or flowers, Thisilyn, or Chaparral root; second, administering Golden Seal root, beet root, and one of the following: Chaparral leaves or flowers, Thisilyn, or Chaparral root at a time interval of at least 30 minutes after the first step; third, administering Golden Seal root, beet root, and one of the following: Chaparral leaves or flowers, Thisilyn, or Chaparral root at a time interval of at least thirty minutes after the second step; and fourth, administering Pure Psyllium husk, Golden Seal root, beet root, activated charcoal, and one of the following: Chaparral leaves or flowers, Thisilyn, or Chaparral root at a time interval of at least thirty minutes after the third step. Preferably, the second step time interval comprises between two and twelve hours. Preferably, the third step time interval comprises between two and twelve hours. Preferably, the fourth step time interval comprises between two and twelve hours.

The present invention also includes a kit for removing accumulated substances from the body comprising at least two liver/blood/kidney cleansers, a lower gastrointestinal tract cleanser, and an adsorbent material. The kit preferably also includes a bulk former, and may further include instructions, wherein the instructions comprise information selected from the group consisting of, time intervals, dosages, substitutes, optional additional components, and dietary guidelines.

### DETAILED DESCRIPTION OF THE PREFERRED EMBODIMENT

The invention relates to a method of removing pharmaceutically active compounds from the human body. The invention contemplates the introduction of a combination of herbs and natural products into the body concurrently with ample amounts of water at intervals effective to rid the body of unwanted chemical substances.

An herb is defined as a plant or a plant part valued for its medicinal, savory, or aromatic qualities, and a nonlimiting list of useful herbs is provided herein. Note that in the present disclosure and claims, the recitation of the herb itself is also considered to cover purified extracts of the herb or an active ingredient of that herb. Active ingredients of most of the herbs recited herein have been identified, and such information is widely available in the literature. For example, it is well established that an active ingredient of Chaparral is nordihydroguaiaretic acid (NDGA), has potent anti-inflammatory activity, possibly due to its ability to block the actions of the enzyme lipoxygenase. Thus, for example, in the present invention, a recitation of Chaparral is considered to also cover the use of nordihydroguaiaretic acid.

The herbs used in this invention include at least two liver/blood/kidney cleansers, a lower gastrointestinal tract cleanser, and an adsorbent material. Preferred liver/blood/kidney cleansers are, for example, Golden Seal root, beet root, and one the following: Chaparral leaves or flowers, Thisilyn, or Chaparral root. A preferred lower gastrointestinal tract cleanser is beet root, and preferred adsorbent materials are ion traps, cationic or anionic ion exchange resins or charcoal, including activated charcoal. In some embodiments, a bulk former is administered. The bulk former can be any kind of roughage or edible fibrous material, such as, for example, Psyllium husk. Administration of these herbs results in the body's reduced ability to absorb ingested substances.

Although the inventor's beliefs relating to the pharmacological and physical activities of the various ingredients are set forth herein, it should be understood that this in no way is intended to limit the invention to a particular theory of operation.

Pharmaceutically-active compounds introduced into the body typically do not form lasting bonds with tissues. Such compounds are typically metabolized, e.g., by enzymes in the liver and elsewhere, and are eventually eliminated from the body. Nevertheless, this natural elimination process is often insufficient to avoid poisoning or harmful or unwanted effects due to the accidental or deliberate ingestion or use of legal or illegal pharmacologically-active agents. Such agents, or their metabolites, can linger in the body for days or even weeks, particularly in the case of fat-soluble compounds, which tend to accumulate in fatty tissue and are eliminated only slowly. For example, tetrahydrocannabinol and its metabolites tend to be stored in the body's fatty tissues long after the compound's initial effect has worn off. It is the purpose of this invention to provide a method of removing unwanted substances, whether synthetic or naturally occurring, from the human body in the shortest possible time by using natural herbs (or their active ingredients).

Chaparral root is a preferred blood/liver/kidney cleanser herb. The availability of this herb, however, is limited. Other effective blood/liver/kidney cleansers in this category include Chaparral leaves or flowers and Thisilyn. Chaparral leaves or flowers can be found in drug stores. The combination of the following: Yellow Dock (Rumex crispus) (root), Red Clover (Trifolium pratense) (blossom), Rosemary (Rosmarinus officinalis) (aerial), and Yucca (Yucca schidigera) (stalk) can provide a substitute for Chaparral leaves or flowers. Likewise, if Golden Seal root cannot be obtained, Golden Seal Herb can be used as its substitute for the purposes of this invention.

Pure Psyllium husk is a type of bulk former or roughage preferably taken before the other herbs in order to, among other things, stimulate bowel movement. Golden Seal root, Chaparral leaves or flowers, Thisilyn, Chaparral root, and beet root work to cleanse the blood, liver, and kidneys of foreign pharmaceutical compounds, beet root acts to cleanse the lower gastrointestinal tract. activated charcoal acts as an adsorbent material, and has a well-established ability to sequester small organic molecules. The above-referenced herbs and their active ingredients are believed to act by binding to the foreign pharmaceutical compounds preventing them from being absorbed into the fatty tissues of the body, and/or by affecting the natural biological pathways by which drugs and metabolites are eliminated from the body. Additionally, the herbs are optimally taken along with ample amounts of water. Water acts to flush the body of the herb-bound pharmaceutical substances.

The water administered in this method can be any water, but is preferably bottled water, distilled water, or deionized or demineralized water. The minerals in tap water are believed to interact with the active ingredients in at least some herbs to reduce their effectiveness. The water is ingested in ample amounts in order to hydrate the tissues, facilitate transport of the active ingredients of the herbs, and facilitate eliminating substances through the body's natural functions. Administration of more than eight ounces of water per dose is suggested. Preferably, thirty-two ounces of water per dose, or more, will be administered.

The effective dose of the herbs is administered within a range depending generally upon a person's weight and percentage of body fat. Any interested individual will be able to determine whether he or she resides at the upper or lower end of the spectrum of average weight and thus the upper or lower end of the range for effective herbal dosage. A typical dose of Golden Seal root ranges from about 200 milligrams to 700 milligrams. The preferred dose of Golden Seal root is about 535 milligrams. A typical dose of any one of the following: Chaparral leaves or flowers, Thisilyn, or Chaparral root ranges from about 120 milligrams to 800 milligrams. The preferred dose of any one of the following: Chaparral leaves or flowers, Thisilyn, or Chaparral root is about 450 milligrams. A typical dose of beet root ranges from about 250 milligrams to 900 milligrams. The preferred dose of beet root is about 605 milligrams. A typical dose of charcoal ranges from about 50 milligrams to 600 milligrams. The preferred dose of charcoal is about 280 milligrams. Note that dosages of active ingredients isolated from the natural herb should reflect the amount of that active ingredient in the specified dosages of the herb per se. Preferably, activated charcoal is used in the dosage between 50 milligrams to 600 milligrams with the preferred dose at about 280 milligrams.

The herbs are taken in varying combinations, preferably at timed intervals. The invention provides of a method of removing pharmaceutically-active substances, including small organic molecules, from the body by first administering Pure Psyllium husk, Golden Seal root, beet root, activated charcoal, and one of the following: Chaparral leaves or flowers, Thisilyn, or Chaparral root. Two to twelve hours later the second dose is administered. Preferably, the second dose is administered at about six hours after the first dose. Second, one administers Golden Seal root, beet root, and one of the following: Chaparral leaves or flowers, Thisilyn, or Chaparral root. Two to twelve hours later the third dose is administered. Preferably, the third dose is administered about six hours after the second dose. Third, one administers Golden Seal root, beet root, and one of the following: Chaparral leaves or flowers, Thisilyn, or Chaparral root. Two to twelve hours later the fourth dose is administered. Preferably, the fourth dose is administered around six hours after the third dose. And fourth, one administers Psyllium husk, Golden Seal root, beet root, activated charcoal, and one of the following: Chaparral leaves or flowers, Thisilyn, or Chaparral root. The timed intervals are approximations, however, administration of the four steps is preferably conducted as close to six hour intervals as possible to ensure the herb levels are maintained and working in the body's tissues. This administration scheme is repeated for two to ten days.

In one embodiment of the invention, it is recommended that the user refrain from eating pork or any kind of animal fat that may inhibit the herbs and clog the circulatory system. Likewise, processed meats such as bologna, salami, hot dogs, etc. are preferably avoided. Fast food, greasy fried food, sugar, chocolate, and dairy products such as milk, cheese, butter, and ice cream, etc. are also preferably avoided. Optimally, no other medications, herbs, vitamin or mineral supplements are be taken; this includes aspirin, cold medications, vitamins A, B, C, E, etc. The possibilities of drug interactions are well recognized, although not filly elucidated by the medical profession, and thus caution advises that potential interactions with the present therapy be avoided to assure safe and effective use of the present invention. It is also recommended that the subject avoid consumption of tap water, soda, coffee, tea, alcohol, or high acid citrus juice during the 5-10 days of herb administration.

To optimize the functions of these herbs it is recommended that the user eat any fresh fruits and vegetables, unsweetened cereals, small amounts of very lean fresh veal, lamb, beef prepared by charbroil, baked fresh fish, baked or broiled skinless chicken or turkey, an egg or two every couple of days, nuts, seeds, moderate amounts of fruit and vegetable juices and plenty of pure water.

What is claimed is:

1. A method of reducing the amount of an exogenous pharmacologically-active compound or its metabolite from the body, comprising:
administering an effective dose of:
at least two liver/blood/kidney cleansers each having the pharmacological activity of at least one of Chaparral, Thisilyn, beet root, Yellow Dock, Clover, Rosemary, Golden Seal, and/or Yucca,
a lower gastrointestinal tract cleanser having the pharmacological activity of beet root; and
an adsorbent material.

2. The method of claim 1, wherein the liver/blood/kidney cleansers are selected from the group consisting of Golden Seal root, Golden Seal Herb, beet root, Chaparral leaves or flowers, Thisilyn, Chaparral root, Yellow Dock (Rumex crispus) (root), Red Clover (Trifolium pratense) (blossom), Rosemary (Rosmarinus officinalis) (aerial), and Yucca (Yucca schidigera) (stalk).

3. The method of claim 1, wherein the lower gastrointestinal tract cleanser comprises beet root.

4. The method of claim 1, wherein the adsorbent material comprises charcoal.

5. The method of claim 4, wherein the charcoal comprises activated charcoal.

6. A method of reducing the amount of an exogenous pharmacologically-active compound or its metabolite from the body, comprising administering to a subject an effective amount of:
(a) Golden Seal root;
(b) one of the following: Chaparral leaves or flowers, Thisilyn, or Chaparral root;
(c) beet root; and
(d) charcoal.

7. The method of claim 6, wherein the charcoal comprises activated charcoal.

8. The method of claim 6, wherein administration comprises oral ingestion.

9. The method of claim 6, further comprising administration of a bulk former.

10. The method of claim 9, wherein the bulk former comprises soluble or insoluble dietary fiber.

11. The method of claim 9, wherein the bulk former comprises Psyllium fiber.

12. The method of claim 6, wherein the administration step is repeated at a multi-hourly interval for a period of about two to ten days.

13. The method of claim 12, wherein the interval ranges from about 2 to about 12 hours.

14. The method of claim 13, wherein the interval is about 6 hours.

15. The method of claim 6, further comprising administration of a volume of water in conjunction with the administration step.

16. The method of claim 15, wherein the water is selected from the group comprising distilled water, bottled water, and demineralized water.

17. The method of claim 15, wherein the volume of water comprises at least about 8 ounces per dose.

18. The method of claim 17, wherein the volume of water comprises about 32 ounces per dose.

19. The method of claim 6, wherein the dose in (a) is about 200 milligrams to about 700 milligrams.

20. The method of claim 19, wherein the dose in (a) is about 535 milligrams.

21. The method of claim 6, wherein the dose in (b) is about 120 milligrams to about 800 milligrams.

22. The method of claim 21, wherein the dose in (b) is about 450 milligrams.

23. The method of claim 6, wherein the dose in (c) is about 250 milligrams to about 900 milligrams.

24. The method of claim 23, wherein the dose in (c) is about 605 milligrams.

25. The method of claim 6 wherein the dose in (d) is about 50 milligrams to about 600 milligrams.

26. The method of claim 25, wherein the dose in (b) is about 280 milligrams.

27. A method of removing exogenous pharmacologically-active substances or their metabolites from the body, comprising the steps of:
first, administering Psyllium fiber, Golden Seal root, beet root, activated charcoal, and one of the following: Chaparral leaves or flowers, Thisilyn, or Chaparral root;
second, administering Golden Seal root, beet root, and one of the following: Chaparral leaves or flowers, Thisilyn, or Chaparral root at a time interval of at least 30 minutes after the first step;
third, administering Golden Seal root, beet root, and one of the following: Chaparral leaves or flowers, Thisilyn, or Chaparral root at a time interval of at least thirty minutes after the second step; and
fourth, administering Pure Psyllium husk, Golden Seal root, beet root, activated charcoal, and one of the following: Chaparral leaves or flowers, Thisilyn, or Chaparral root at a time interval of at least thirty minutes after the third step.

28. The method of claim 27, wherein the second step time interval comprises between two and twelve hours.

29. The method of claim 27, wherein the third step time interval comprises between two and twelve hours.

30. The method of claim 27, wherein the fourth step time interval comprises between two and twelve hours.

31. A kit for removing accumulated substances from the body comprising at least two liver/blood/kidney cleansers, a lower gastrointestinal tract cleanser, and an adsorbent material.

32. The kit of claim 31, further comprising a bulk former.

33. The kit of claim 32, further comprising instructions, wherein the instructions comprise information selected from the group consisting of, time intervals, dosages, substitutes, optional additional components, and dietary guidelines.
